(19) United States
(12) Patent Application Publication
(10) Pub. No.: US 2023/0419503 A1
(43) Pub. Date: Dec. 28, 2023

(54) SYSTEM AND METHOD FOR OPERATING ROOM HUMAN TRAFFIC MONITORING

(71) Applicant: SURGICAL SAFETY TECHNOLOGIES INC., Toronto (CA)

(72) Inventors: Frank RUDZICZ, Toronto (CA); Amar S. CHAUDHRY, Toronto (CA); Shuja KHALID, Toronto (CA); Teodor Pantchev GRANTCHAROV, Stouffville (CA); Tianbao LI, Toronto (CA)

(21) Appl. No.: 18/037,987

(22) PCT Filed: Nov. 19, 2021
(86) PCT No.: PCT/CA2021/051649

§ 371 Date: May 19, 2023

Related U.S. Application Data
(60) Provisional application No. 63/115,839, filed on Nov. 19, 2020.

(51) Int. Cl. G06T 7/20

(52) U.S. Cl. CPC G06T 7/20; G06T 2207/10016; G06T 2207/20084; G06T 2207/20076; G06T 2207/20081; G06T 2207/30196; G06T 2207/30242

(57) ABSTRACT

Systems and methods for traffic monitoring in an operating room are disclosed herein. Video data of an operating room is received, the video data captured by a camera having a field of view for viewing movement of a plurality of individuals in the operating room during a medical procedure. An event data model is stored, the model including data defining a plurality of possible events within the operating room is stored. The video data is processed to track movement of objects within the operating room, the objects including at least one body part, and the processing using at least one detector trained to detect a given type of the objects. A likely occurrence of one of the possible events is determined based on the tracked movement.

### CROSS-REFERENCE

This application claims priority to and benefits of U.S. Provisional Patent Application No. 63/115,839, filed on Nov. 19, 2020, the entire content of which is herein incorporated by reference.

### FIELD

The present disclosure generally relates to the field of video processing, object detection, and object recognition.

### BACKGROUND

Embodiments described herein relate to the field of medical devices, systems and methods and, more particularly, to medical or surgical devices, systems, methods and computer readable media to monitor activity in an operating room (OR) setting or patient intervention area.

### SUMMARY

In accordance with an aspect, there is provided a computer-implemented method for traffic monitoring in an operating room. The method includes: receiving video data of an operating room, the video data captured by a camera having a field of view for viewing movement of a plurality of individuals in the operating room during a medical procedure; storing an event data model including data defining a plurality of possible events within the operating room; processing the video data to track movement of objects within the operating room, the objects including at least one body part, and the processing using at least one detector trained to detect a given type of the objects; and determining a likely occurrence of one of the possible events based on the tracked movement.

In some embodiments, the at least one body part includes at least one of a limb, a hand, a head, or a torso.

In some embodiments, the plurality of possible events includes adverse events.

In some embodiments, the method may further include determining a count of individuals based on the processing using at least one detector.

In some embodiments, determining a likely occurrence of one of the possible events includes determining that the count of individuals exceeds a pre-defined threshold.

In some embodiments, the count describes a number of individuals in the operating room.

In some embodiments, the count describes a number of individuals in a portion of the operating room.

In some embodiments, the method may further include determining a correlation between the likely occurrence of one of the possible events and a distraction.

In some embodiments, the objects include a device within the operating room.

In some embodiments, the device is a radiation-emitting device.

In some embodiments, the device is a robotic device.

In some embodiments, the at least one detector includes a detector trained to detect said robotic device.

In some embodiments, the method may further include storing a floorplan data structure.

In some embodiments, the floorplan data structure includes data defining at least one sterile field and at least one non-sterile field in the operating room.

In some embodiments, the floorplan data structure includes data defining a 3D model of at least a portion of the operating room.

In some embodiments, the determining the likely occurrence of one of the possible adverse events is based on the tracked movement of at least one of the objects through the at least one sterile field and the at least one non-sterile field.

In accordance with another aspect, there is provided a computer system for traffic monitoring in an operating room. The system includes a memory; a processor coupled to the memory programmed with executable instructions for causing the processor to: receive video data of an operating room, the video data captured by a camera having a field of view for viewing movement of a plurality of individuals in the operating room during a medical procedure; store an event data model including data defining a plurality of possible events within the operating room; process the video data to track movement of objects within the operating room, the objects including at least one body part, and the processing using at least one detector trained to detect a given type of the objects; and determine a likely occurrence of one of the possible events based on the tracked movement.

In some embodiments, the at least one body part includes at least one of a limb, a hand, a head, or a torso.

In some embodiments, the plurality of possible events includes adverse events.

In some embodiments, the instructions may further cause the processor to determine a count of individuals based on the processing using at least one detector.

In some embodiments, determining a likely occurrence of one of the possible events includes determining that the count of individuals exceeds a pre-defined threshold.

In some embodiments, the count describes a number of individuals in the operating room.

In some embodiments, the count describes a number of individuals in a portion of the operating room.

In some embodiments, the instructions may further cause the processor to determine a correlation between the likely occurrence of one of the possible events and a distraction.

In some embodiments, the objects include a device within the operating room.

In some embodiments, the device is a radiation-emitting device.

In some embodiments, the device is a robotic device.

In some embodiments, the at least one detector includes a detector trained to detect said robotic device.

In some embodiments, the instructions may further cause the processor to store a floorplan data structure.

In some embodiments, the floorplan data structure includes data defining at least one sterile field and at least one non-sterile field in the operating room.

In some embodiments, the floorplan data structure includes data defining a 3D model of at least a portion of the operating room.

In accordance with yet another aspect, there is provided an non-transitory computer-readable storage medium storing instructions which when executed adapt at least one computing device to: receive video data of an operating room, the video data captured by a camera having a field of view for viewing movement of a plurality of individuals in the operating room during a medical procedure; store an event data model including data defining a plurality of possible events within the operating room; process the video data to track movement of objects within the operating room, the objects including at least one body part, and the processing using at least one detector trained to detect a given type of the objects; and determine a likely occurrence of one of the possible events based on the tracked movement.

In accordance with still another aspect, there is provided a system for generating de-identified video data for human traffic monitoring in an operating room. The system includes a memory; and a processor coupled to the memory programmed with executable instructions for causing the processor to: process video data to generate a detection file with data indicating detected heads, hands, or bodies within the video data, the video data capturing activity in the operating room; compute regions corresponding to detected heads, hands, or bodies in the video data using the detection file; for each region corresponding to a detected head, hand, or body generate blurred, scrambled, or obfuscated video data corresponding to that detected region; generate de-identified video data by integrating the video data and the blurred, scrambled, or obfuscated video data; and output the de-identified video data to the memory or an interface application.

In some embodiments, the processor is configured to generate a frame detection list indicating head, hand, or body detection data for each frame of the video data, the detection data indicating one or more regions corresponding to one or more detected heads, hands, or bodies in the respective frame.

In some embodiments, the processor is configured to use a model architecture and feature extractor to detect features corresponding to the heads, hands, or bodies within the video data.

In some embodiments, the processor is configured to generate the de-identified video data by, for at least one frame in the video data, creating a blurred, scrambled, or obfuscated region in a respective frame corresponding to a detected head, hand, or body in the respective frame.

In some embodiments, the processor is configured to compare a length of the video data with a length of the de-identified video data.

In some embodiments, the processor is configured to compute regions corresponding to detected heads, hands, or bodies in the video data by, for each batch of frames of the video data, run an inference session to compute a region for each detected head, hand, or body in the respective batch of frames and compute a confidence score for the respective region, wherein the processor adds the computed regions and confidence scores to a detection class list.

In some embodiments, the processor is configured to compute head, hand, or body count data based on the detected heads, hands, or bodies in the video data, and output the count data, the count data comprising change in head, hand, or body count data over the video data.

In some embodiments, the processor is configured to compute head, hand, or body count data by computing that count data based on the detected heads, hands, or bodies for each frame of the video data, and to compute the change in head, hand, or body count data over the video data by comparing the head, hand, or body count data for the frames of the video data, each computed change in count having a corresponding time in the video data.

In some embodiments, the processor is configured to compute timing data for each change in head, hand, or body count in the change in head, hand, or body count data over the video data.

In some embodiments, the processor is configured to compute a number of people in the operating room based on the detected heads, hands, or bodies in the video data.

In some embodiments, the processor is configured to, for one or more regions corresponding to a detected head, hand, or body, compute a bounding box or pixel-level mask for the respective region, a confidence score for the detected head, hand, or body, and compute data indicating the bounding boxes or pixel-level masks, the confidence scores, and the frames of the video data.

In accordance with another aspect, there is provided a system for monitoring human traffic in an operating room. The system includes a memory; a processor coupled to the memory programmed with executable instructions, the instructions configuring an interface for receiving video data comprising data defining heads, hands, or bodies in the operating room; and an operating room monitor for collecting the video data from sensors positioned to capture activity of the heads, hands, or bodies in the operating room and a transmitter for transmitting the video data to the interface. The instructions configure the processor to: compute regions corresponding to detected heads, hands, or bodies in the video data using a feature extractor and detector to extract and process features corresponding to the heads, hands, or bodies within the video data; generate head, hand, or body detection data by automatically tracking the regions corresponding to a detected head, hand, or body across frames of the video data; generate traffic data for the operating room using the head, hand, or body detection data and identification data for the operating room; and output the traffic data.

In some embodiments, the processor is configured to generate a frame detection list indicating head, hand, or body detection data for each frame of the video data, the detection data indicating one or more regions corresponding to one or more detected heads, hands, or bodies in the respective frame.

In some embodiments, the processor is configured to compute regions corresponding to detected heads, hands, or bodies in the video data by, for each batch of frames of the video data, run an inference session to compute a region for each detected head, hand, or body in the respective batch of frames and compute a confidence score for the respective region, wherein the processor adds the computed regions and confidence scores to a detection class list.

In some embodiments, the processor is configured to compute head, hand, or body count data based on the detected heads, hands, or bodies in the video data, and output the count data, the count data comprising change in head, hand, or body count data over the video data.

In some embodiments, the processor is configured to compute head, hand, or body count data by computing that count data based on the detected heads, hands, or bodies for each frame of the video data, and to compute the change in head, hand, or body count data over the video data by comparing the head, hand, or body count data for the frames of the video data, each computed change in count having a corresponding time in the video data.

In some embodiments, the processor is configured to compute timing data for each change in head, hand, or body count in the change in head, hand, or body count data over the video data.

In some embodiments, the processor is configured to compute a number of people in the operating room based on the detected heads, hands, or bodies in the video data.

In some embodiments, the processor is configured to, for one or more regions corresponding to a detected head, hand, or body, compute a bounding box or pixel-level mask for the respective region, a confidence score for the detected head, hand, or body, and compute data indicating the bounding boxes or pixel-level masks, the confidence scores, and the frames of the video data.

In accordance with another aspect, there is provided a process for displaying traffic data for activity in an operating room on a graphical user interface (GUI) of a computer system. The process includes: receiving via the GUI a user selection to display video data of activity in the operating room; determining traffic data for the video data using a processor with a detector that tracks regions corresponding to detected heads, hands, or bodies in the video data; automatically displaying or updating visual elements integrated with the displayed video data to correspond to the tracked regions corresponding to detected heads, hands, or bodies in the video data; receiving user feedback from the GUI for the displayed visual elements, the feedback confirming or denying a detected head, hand, or body; and updating the detector based on the feedback.

In accordance with another aspect, there is provided a system for human traffic monitoring in the operating room. The system has a server having one or more non-transitory computer readable storage media with executable instructions for causing a processor to: process video data to detect heads, hands, or bodies within video data capturing activity in the operating room; compute regions corresponding to detected areas in the video data; for each region corresponding to a detected head, generate blurred, scrambled, or obfuscated video data corresponding to a detected head; generate de-identified video data by integrating the video data and the blurred, scrambled, or obstructed video data; and output the de-identified video data.

In some embodiments, the processor is configured to generate a frame detection list indicating head detection data for each frame of the video data, the head detection data indicating one or more regions corresponding to one or more detected heads in the respective frame.

In some embodiments, the processor is configured to use a model architecture and feature extractor to detect the heads, hands, or bodies within the video data.

In some embodiments, the processor is configured to generate the de-identified video data by, for each frame in the video data, creating a blurred copy of the respective frame, for each detected head in the respective frame, replacing a region of the detected head in the respective frame with a corresponding region in the blurred copy of the respective frame.

In some embodiments, the processor is configured to compare a length of the video data with a length of the de-identified video data.

In some embodiments, the processor is configured to compute regions corresponding to detected heads, hands, or bodies in the video data by, for each batch of frames of the video data, running an inference session to compute a region for each detected head, hand, or body in the respective batch of frames and compute a confidence score for the respective region, wherein the processor adds the computed regions and confidence scores to a detection class list.

In some embodiments, the processor is configured to compute head, hand, or body count data based on the detected regions in the video data, and output those count data, comprising change in count data over the video data.

In some embodiments, the processor is configured to compute head, hand, or body count data by, computing count data based on the detected regions for each frame of the video data, and compute the change in head, hand, or body count data over the video data by comparing the count data for the frames of the video data, each computed change in count having a corresponding time in the video data.

In some embodiments, the processor is configured to compute timing data for each change in head, hand, or body count in the change in count data over the video data.

In some embodiments, the processor is configured to compute a number of people in the operating room based on the detected heads, hands, or bodies in the video data.

In some embodiments, the processor is configured to, for each region corresponding to a detected head, hand, or body, compute a bounding box or pixel-level mask for the respective region, a confidence score for the detected region, and a frame of the video data, and compute data indicating the bounding boxes, the confidence scores and the frames of the video data.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

FIG. 1 illustrates a platform for operating room (OR) human traffic monitoring according to some embodiments.

FIG. 2 illustrates a workflow diagram of a process for OR human traffic monitoring according to some embodiments.

FIG. 3 illustrates a workflow diagram of a process for OR human traffic monitoring according to some embodiments.

FIG. 4 illustrates a workflow diagram of a process for head blurring in video data according to some embodiments.

FIG. 5 illustrates a workflow diagram of a process for head detection in video data according to some embodiments.

FIG. 6 illustrates a graph relating to local extrema.

FIG. 7 illustrates a schematic of an architectural platform for data collection in a live OR setting or patient intervention area according to some embodiments.

FIG. 8 illustrates an example process in respect of learning features, using a series of linear transformations.

FIG. 9A illustrates experimental results of an example system used to de-identify features from a video obtained at a first hospital site.

FIG. 9B illustrates experimental results of an example system used to de-identify features in from a video obtained at a second hospital site.

FIG. 10A illustrates experimental results of an example system used to de-identify features from the video obtained at a first hospital site using different sampling rates.

FIG. 10B illustrates experimental results of an example system used to de-identify features in from a video obtained at a second hospital site using different sampling rates.

FIG. 11 illustrates example processing time of various de-identification approach types in hours in a first chart.

FIG. 12 illustrates example processing time of various de-identification approach types in hours in a second chart.

### DETAILED DESCRIPTION

Embodiments of methods, systems, and apparatus are described through reference to the drawings.

Embodiments may provide a system, method, platform, device, and/or computer readable medium for monitoring patient activity in a surgical operating room (OR), intensive care unit, trauma room, emergency department, interventional suite, endoscopy suite, obstetrical suite, and/or medical or surgical ward, outpatient medical facility, clinical site, or healthcare training facility (simulation centres). These different example environments or settings may be referred to as an operating or clinical site.

Embodiments described herein may provide devices, systems, methods, and/or computer readable medium for operating room human traffic monitoring.

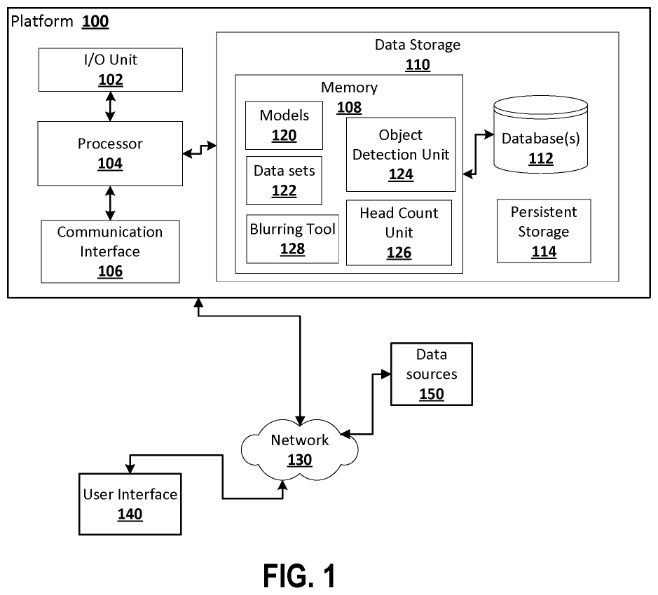

FIG. 1 is a diagram of a platform 100 for operating room (OR) human traffic monitoring. The platform 100 can detect heads in video data capturing activity in an operating room. The platform 100 can compute regions of the video data corresponding to detected heads. The platform 100 can determine changes in head, hand, or body count. The platform 100 can generate de-identified video data by using blurred video data for the regions of the video data corresponding to detected heads. The platform 100 can output de-identified video along with other computed data. In an embodiment, the platform is configured for detecting body parts (e.g., heads) and changes in counts of the body parts, and the changes are used to generate output insight data sets relating to human movement or behaviour.

The platform 100 can provide real-time feedback on the number of people that are in the operating room for a time frame or range by processing video data. Additionally, in some embodiments, the system 100 can anonymize the identity of each person by blurring, scrambling, or obstructing their heads in the video data. The platform 100 can generate output data relating to operating room human traffic monitoring to be used for evaluating efficiency and/or ergonomics, for example.

Extracting head-counts from video recordings can involve manual detection and annotation of the number of people in the OR which can be time-consuming. Further, blurring, scrambling, or obfuscating heads is also a manual procedure. This can be very time consuming for analysts. Some approaches might only detect faces, and only when there are no severe obstructions (i.e., not covered by objects like masks). In particular, some approaches focus on the detection of contours and contrasts created by the eyebrows, eyes, nose and mouth. This can be problematic in the case of the OR, where individuals have masks and caps on, and where the count needs to account for everyone. Platform 100 can implement automatic human traffic monitoring in the OR using object detection and object recognition, and can accommodate obstructions, such as masks, for example.

The platform 100 connects to data sources 170 (including one or more cameras, for example) using network 130. The platform 100 can receive video data capturing activity in an OR. Network 130 (or multiple networks) is capable of carrying data and can involve wired connections, wireless connections, or a combination thereof. Network 130 may involve different network communication technologies, standards and protocols, for example. User interface 140 application can display an interface of visual elements that can represent de-identified video data, head count metrics, head detection data, and alerts, for example. The visual elements can relate to head, hand, or body detection and count data linked to adverse events, for example.

In some embodiments, the video data is captured by a camera having an angle of view suitable for imaging movement of a plurality of individuals in the operating room during a medical procedure. Video data may, for example, be captured by a wide angle-of-view camera suitable for imaging a significant portion of an operating room (e.g., having a suitable focal length and sensor size). Video data may also, for example, be captured by a plurality of cameras each suitable for imaging a fraction of an operating room. Video data may also, for example, be captured by a plurality of cameras operating in tandem and placed to facilitate 3D reconstruction from stereo images.

The platform 100 can include an I/O Unit 102, a processor 104, communication interface 106, and data storage 110. The processor 104 can execute instructions in memory 108 to implement aspects of processes described herein. The processor 104 can execute instructions in memory 108 to configure models 120, data sets 122, object detection unit 124, head count unit 126, blurring tool 128, and other functions described herein. The platform 100 may be software (e.g., code segments compiled into machine code), hardware, embedded firmware, or a combination of software and hardware, according to various embodiments. The models 120 can include architectures and feature extractors for use by object detection unit 124 to detect different objects within the video data, including human heads. The models 120 can be trained using different data sets 122. The models 120 can be trained head detection for use by object detection unit 124 to detect heads within the video data of the OR, for example.

The object detection unit 124 can process video data to detect heads within the video data. The video data can capture activity in the OR including human traffic within the OR. The object detection unit 124 can compute regions corresponding to detected heads in the video data using models 120. The region can be referred to as a bounding box. The region or bounding box can have different shapes. A region corresponds to the location of a detected head within a frame of the video data. The head detection data can be computed by the object detection unit 124 on a per frame basis. In some embodiments, the object detection unit 124 is configured to generate a frame detection list indicating head detection data for each frame of the video data. The head detection data can indicate one or more regions corresponding to one or more detected heads in the respective frame.

In some embodiments, the object detection unit 124 is configured to compute regions corresponding to detected heads, hands, or bodies in the video data by, for each batch of frames of the video data, running an inference session to compute a region for each detected head in the respective batch of frames. The inference session uses the models 120 (and feature extractors) to detect the heads in the video data. The object detection unit 124 can compute a confidence score for each respective region that can indicate how confident that it is a detected head, hand, or body (instead of another object, for example). The object detection unit 124 can add the computed regions and confidence scores to a detection class list.

In some embodiments, the object detection unit 124 is configured to, for each region corresponding to a detected head, hand, or body, compute a bounding box for the respective region, a confidence score for the detected head, and a frame of the video data. The object detection unit 124 can compute data indicating the bounding boxes, the confidence scores and the frames of the video data.

For each region/bounding box corresponding to a detected head, the blurring, scrambling, or obfuscating tool 128 can generate blurred, scrambled, or obfuscated video data corresponding to a detected head. The blurring tool 128 generates and outputs de-identified video data by integrating the video data and the blurred, scrambled, or obfuscated video data. In some embodiments, the blurring, scrambling, or obfuscating tool 128 is configured to generate the de-identified video data by, for each frame in the video data, creating a blurred, scrambled, or obfuscated copy of the respective frame. For each detected head, hand, or body in the respective frame, the tool 128 may be configured to replace a region of the detected region in the respective frame with a corresponding region in the blurred, scrambled, or obfuscated copy of the respective frame. In some embodiments, the tool 128 is configured to compare a length of the video data with a length of the de-identified video data to make sure frames were not lost in the process.

In some embodiments, the head count unit 126 is configured to compute head count data based on the detected heads in the video data, and output the head count data. In some embodiments. The head count unit 126 may be implemented based on a masked, region-based convolutional neural networks (Mask R-CNN), for example, under the Detectron2 framework. This may or may not incorporate explicit knowledge encoding, such as the identification of human forms through key body parts or points (e.g., the shoulders, the elbows, the base of the neck), with or without occlusions. The head count data includes change in head count data over the video data. In some embodiments, the head count unit 126 is configured to compute head count data by, computing head count data based on the detected heads for each frame of the video data, and compute the change in head count data over the video data. The head count unit 126 compares the head count data for the frames of the video data. The head count unit 126 determines, for each computed change in head count, a corresponding time in the video data for the change. That is, in some embodiments, the head count unit 126 is configured to compute timing data for each change in head count in the change in head count data over the video data to indicate when changes in head count occurred over the video. The timing data can also be linked to frame identifiers, for example.

In some embodiments, the platform 100 is configured to compute a number of people in the operating room based on the detected heads in the video data. Subsequently, headcounts may be used as a conditioning variable in various analysis, including room efficiency, level of distractions, phase of the operation, and so on. These analysis can be clinical in nature (e.g., how often people leave and enter the room is related to distractions, which are clinically meaningful, and is obtainable from changes in head counts) or technical (e.g., the number of detected heads informs de-identification algorithms of the number of bodies to obfuscate in the video).

The object detection unit 124 is adapted to implement deep learning models (e.g. R-FCN). The deep learning models can be trained using a dataset constructed from the video feed in the operating rooms (ORs). This dataset can be made up of random frames taken from self-recorded procedures, for example. The training dataset can contain bounding box annotations around each of the heads of people in the operating room. The system 100 can use model(s) and training process to produce the (trained) output model, which is a weights file, that can be used to evaluate any new, unseen, frame. Evaluating video using the trained model 120 can result in two output files. The first output file records changes to the number of people in the room, as well as recording a timestamp of when the head-count change occurred. The second output file contains the bounding boxes for each detection, a confidence score of this detection and the frame.

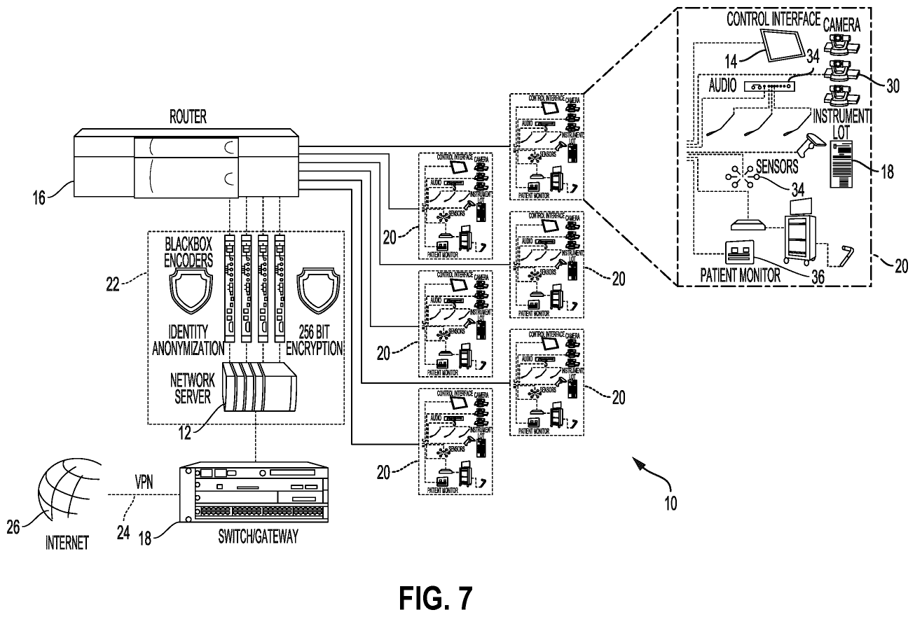

Data from the first file can be used by the platform 100 for the automatic identification of the number of individuals in the OR. Data in the second file allows for the system 100 to update the video data for automatic blurring of faces. Further, this data can be used in statistical models that assess and determine the relationships between the number of individuals in the OR and events of interest in the OR (Including both surgery specific and otherwise). The platform 100 can link the head count and detection data to statistical data computed by the platform 10 described in relation to FIG. 7. The platform 100 can integrate with platform 10 in some embodiments.

In some embodiments, the platform 100 stores an event data model having data defining a plurality of possible events within the OR. The event data model may store data defining, for example, adverse events, other clinically significant events, or other events of interest. Events of interest may include, for example, determining that the number of individuals in the OR (or a portion of the OR) exceeds a pre-defined limit; determining that an individual is proximate to a radiation-emitting device or has remained in proximity of a radiation-emitting device for longer than a pre-defined safety limit; determining that an individual (or other object) has moved between at least one sterile field of the OR and at least one non-sterile field of the OR. The platform 100 may use this event data model to determine a likely occurrence of one of the possible events based on tracked movement of objects in the OR. For example, given the location of a body part (e.g., a head) in the video, the number of frames for which that head remains within a predefined region may be determined, and if the determined number of frames exceeds a pre-defined safety threshold, the body part is determined to be in proximate to a radiation-emitting device for too long.

In some embodiments, the platform 100 maintains a plurality of detectors, each trained to detect a given type of object that might be found in an OR. For example, one or more detectors may be trained to detect objects that are body parts such as a limb, a hand, a head, a torso, or the like. For example, one or more detectors may be trained to detect devices in the OR. Such devices may include stationary devices (e.g., x-ray machines, ultrasound machines, or the like). Such devices may also include mobile devices such as mobile robotic devices (or simply referred to as robotic devices). For example, one or more detectors may be trained to detect other features of interest in the OR such as doors, windows, hand-wash stations, various equipment, or the like.

In some embodiments, the platform 100 stores a floorplan data structure including data that describes a floorplan or layout of at least a portion of the OR. In some embodiments, the floorplan data structure may also include metadata regarding the layout or floorplan of the OR such as, for example, the location of at least one sterile field and at least one non-sterile field in the OR, the location of certain devices or equipment (e.g., devices that might present risk such as radiation sources, points of ingress and egress, etc.). In some embodiments, the floorplan data structure may include data defining a 3D model of at least a portion of the OR with location of objects defined with reference to a 3D coordinate system. In some embodiments, the movement of objects may be tracked within such a 3D coordinate system.

The platform 100 may process the floorplan data structure in combination of detected movement of objects to determine when events of interest may have occurred, e.g., when someone has moved from a non-sterile field to a sterile-field, when someone has entered or left the OR, when someone has moved into proximity to a particular device or equipment, or the like.

The platform 100 implements automatic head detection and is capable of generating detection output in real-time. The object detection unit 124 can implement head detection. The platform 100 can also include person detection and tracking. The movement of a particular de-identified individual can therefore be traced. For each OR, the model can be fine-tuned. The platform 100 can be expanded to include detection of heads outside of the OR, to track movements of the staff in different hospital settings, for example.

Models that are generated specifically for the task of object detection can be trained using video data examples which can include examples from the OR, with occlusions, different coloured caps, masks, and so on. In an experiment, the dataset can include over 10,000 examples with bounding boxes over the heads. Training can be performed for 200,000 iterations. After the training is complete, the model and its weights can be exported to a graph. The exporting of the graph can be performed with a function embedded within a machine learning algorithm. For the tracking of the heads, over a series of frames, the head detection can be run to detect the most probable new location of the objects in the previous frame, by performing geometrical and pixel transformations. For example, a training data set can be generated using video data with heads recorded in the OR. This video data can include heads that were partially obstructed. The training process can update the learning rate so avoid local extrema (e.g. as the model is trained the learning rate gets smaller so it does not get stuck in the local minimum). The model can minimize a loss function (number of heads lost) so it might get stuck in the local minimum but would prefer to obtain global minimum. Reducing the learning rate can make it more feasible for the model to reach the global minimum for convergence.

A training function can be used for training the detection model. The source of the data can be changed to a current dataset file created for head detection, and the model can be pointed towards the model with modified hyper-parameters. The learning rate can be changed over the training process. The variables in the detection model can be changed (e.g. by calling an API function) with a new model and new data set. The model can process video by converting video data into frames per second. An inference graph can be updated so that it can use a high number of frames at a time, implement different processes at the same time, and process different frames at a time. For a production stage, the detection model can work in real-time.

The head count unit 126 implements automatic head count for each moment in time (or video frame) and is capable of generating head count output in real-time. The platform 100 processes video data from OR recordings to determine head count for data extraction. The platform 100 processes video data to update the video data by blurring the faces for anonymity. The platform 100 implements this data extraction to create privacy for OR members. The platform 100 studies statistical relationships to create models to guide, consult and train for future OR procedures.

In some embodiments, the platform 100 determines a count of individuals based on processing video data using one or more detectors. In some embodiments, determining a likely occurrence of one of the possible events includes determining that the count of individuals exceeds a pre-defined threshold. This count may describe a total number of individuals in the OR, or a number of individuals in a portion of the OR.

In some embodiments, the platform 100, may generate reports based on tracked movement. The platform 100 may, for example, generate reports including aggregated data or statistical analysis of tracked movement, e.g., to provide insights on events of interest in the OR, or traffic within the OR. Such reports may be presented by way of a GUI with interactive elements that allow a user to customize the data being aggregated, customize the desired statistical analysis, or the like.

The platform 100 can run an inference on all the frames to compute the bounding boxes and score per box, for each frame of the video (or specified frame rate). Afterwards, all the detections are evaluating, frame by frame. This process includes, counting how many detections occurred per frame, reading the next frame, and comparing if the number of detections has changed. The head counts, and the corresponding video time of the frame, is included in a data file. This file can contain the times and counts at the points where the head count changed in the video feed. The platform 100 process a list for each frame to compute how many heads are in each frame. The platform 100 compares the head count for frames. The platform 100 also keeps track of the time to detects that the head count changes at a particular time/frame (e.g. minute 5 frame 50). The platform 100 can record when the head count change and this can be used to annotate the time line with head count data.

The platform 100 can use these output data streams to construct models involving the relationships between the number of people in the OR and the probability of an event occurring. The platform 100 can use these output data streams to provide real-time feedback in the OR using one or more devices. For example, the platform 100 uses a dataset of OR recordings (which can be captured by platform 10 of FIG. 7) to train the model, as well as hyperparameter tuning. The platform 100 can use a common timeline for statistical analysis. The platform 100 can trigger alerts based on the statistical data. For example, a statistical finding can be that when there are more than 8 people in the OR, the risk of an adverse event can double. The platform 100 can trigger alerts upon determining the number of people in the room. If the computed number exceeds a threshold, then an alert can be triggered. This can help to limit the number of people in the room and avoid adverse events. The statistical analysis can correlate events with distractions, for example. Distractions can be associated with safety concerns. For example, if there is are too many people in the room this can also trigger safety issues. Movement/gestures may also trigger safety issues and these can be computed by platform 100. There can also be auditory distractions, looking at devices, and so on. The platform 100 can provide distraction metrics as feedback. The platform 100 can detect correlations between distractions and events that occur.

The platform 100 can use a common timeline. The platform 100 can detect individuals and track how much they moved. Individuals can be tagged person 1, person 2, person 3, or other de-identified/anonymized identifier that can be used for privacy. Each person or individual can be associated with a class of person and this can be added as a layer of the identifier.

The platform 100 can track movement of objects within the OR, e.g., devices, body parts, etc. The platform 100 can determine a likely occurrence of a possible event, as defined in the event data model, based on the tracked movement.

The platform 100 can provide data acquisition. The platform 100 can detect correlations between events of interest occurring in the OR and the number of people in the OR. This framework can allow an additional measure of safety to be taken during surgical procedures, where the number of people inside the OR is limited to a threshold number (e.g. 8 people). Automatic detection of this information in real-time can allow for more advanced analytical studies of such relationships, real-time feedback, and improved efficiency among other benefits.

The platform 100 implements automatic blurring of people's faces/heads and is capable of operating in real-time. This can provide privacy. The output data can include video data with face blurring which can be beneficial for purpose such as creating a peer-review of the video data while providing privacy. For example, debriefing OR staff with quality improvement reports containing de-identified members of the staff ensures anonymity that makes clinicians more receptive to constructive feedback. Positive reception to feedback improves the probability for successful implementation of training initiatives aimed at improving skills/performance of OR staff. Once the heads are detected, the platform 100 can process video data to update the video data by blurring the faces for anonymity. The platform 100 can implement the blurring as a post-processing step. A script can go through all the frames in the video and blur each of the detections per frame. The platform 100 can store each frame into a new video. Once all the frames are completed, an command can be called so that the audio stream included in the original video can be multiplexed with the blurred video. The platform 100 can run the detection process on the whole video and use a frame detection process to outputs boxes on frames of the video data and corresponding (confidence) scores, along with frames. A program can read the output and, for each frame, implement the blurring based on a threshold confidence score. When the platform 100 finishes blurring for the frame it can add the blurred frame to a new video and add the soundtrack from the original video. The threshold score can be static or learned or modified as a configuration/user.

The platform 100 can use different models. For example, the platform 100 can use a pre-trained model on non-specialized workers (specialized workers being surgeons and nurses) and expanding this model with data of surgeons, or GAN generated data. Training a different model with this data can also be used. The platform 100 can use computer vision algorithms. Such examples include using transfer learning plus a classifier or a Support Vector Machines, Nearest Neighbor, and so on.

The I/O unit 102 can enable the platform 100 to interconnect with one or more input devices, such as a keyboard, mouse, camera, touch screen and a microphone, and/or with one or more output devices such as a display screen and a speaker.

The processor 104 can be, for example, a microprocessor or microcontroller, a digital signal processing (DSP) processor, an integrated circuit, a field programmable gate array (FPGA), a reconfigurable processor, or various combinations thereof.

Memory 108 may include a suitable combination of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Data storage devices 110 can include memory 108, databases 112 (e.g. graph database), and persistent storage 114.

The communication interface 106 can enable the platform 100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including various combinations of these.

The platform 100 can be operable to register and authenticate users (using a login, unique identifier, and password for example) prior to providing access to applications, a local network, network resources, other networks and network security devices. The platform 100 can connect to different machines or entities (e.g. data sources 150).

The data storage 110 may be configured to store information associated with or created by the platform 100. The data storage 110 can store raw video data, head detection data, count data, and so on. The data storage 110 can implement databases, for example. Storage 110 and/or persistent storage 114 may be provided using various types of storage technologies, such as solid state drives, hard disk drives, flash memory, and may be stored in various formats, such as relational databases, non-relational databases, flat files, spreadsheets, extended markup files, and so on

The platform 100 can be used for expert analysts. For this stakeholder, the platform 100 serves the purpose of identifying the portions of the surgical case in which the number of people in the room have changed (increased or decreased) potentially past critical numbers. This helps identify particular moments of interest in the case. This could be indicative of a critical moment where external help was requested by the surgical team because of an adverse event.

The platform 100 can be used for clients. For this stakeholder, the platform 100 serves the purpose of anonymizing the video. As part of the solution report presented to each client, segments of video might be made available for them to refresh their memory on what had occurred, and use for training purposes. By blurring the heads of the staff present in the video, the platform 100 can maintain the non-punitive nature of the content, and re-enforcing education purposes.

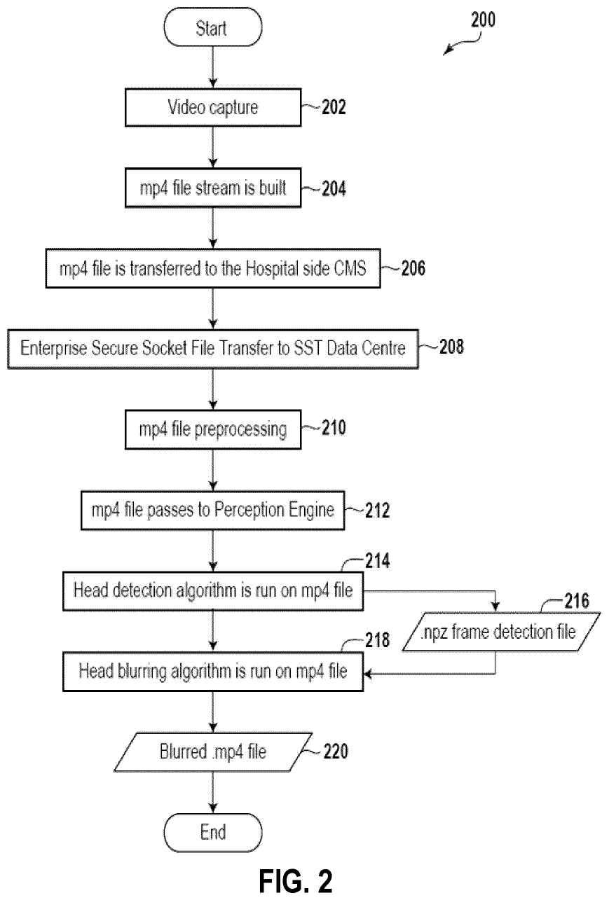

FIG. 2 illustrates a workflow diagram of a process 200 for OR human traffic monitoring according to some embodiments. The process 200 involves detecting heads in video data. At 202, video data from the OR is captured using video cameras, for example. Other data from the OR can also be captured using different types of sensors. At 204, a video file stream (including the OR video data) is built. At 206, the file stream can be transferred to an interface system within a health care facility. At 208, the file stream can be transferred via an enterprise secure socket filed transfer to a data centre and/or platform 100. At 210, the file stream in pre-processed. At 212, the platform 100 receives the file stream (which can also be referred to as a perception engine). At 214, the object detection unit 124 processes the file stream to detect heads and/or other objects in the video data. At 216, the object detection unit 124 generates a frame detection file that includes head detection data. The detection data can be on a per frame basis. The detection data can also be linked to timing data. The frame detection file can also include head count data (e.g. as generated by head count unit 126). The frame detection file can also include boxes annotating the video data to define each detected head. At 218, the blurring tool 128 implements blurring of the detected head. At 220, the blurred head video file is provided as output (e.g., as a blurred .mp4 file transformed from the original .mp4 file stream).

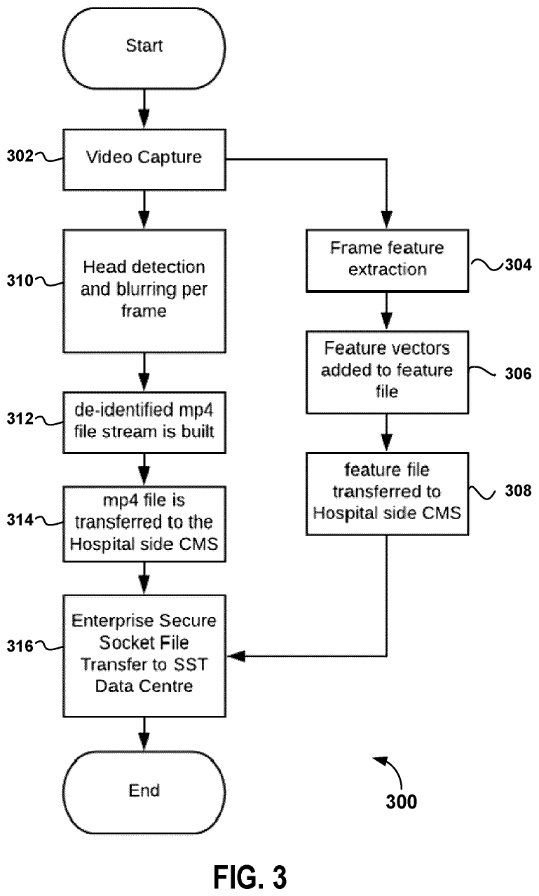

FIG. 3 illustrates a workflow diagram of a process 300 for OR human traffic monitoring according to some embodiments. The process 300 involves detecting heads in video data. The process 300 involves de-identification by design. At 302, video data from the OR is captured using video cameras, for example. Other data from the OR can also be captured using different types of sensors. At 304, the object detection unit 124 implements frame feature extraction. At 306, the object detection unit 124 generates feature vectors which is added to the feature file. At 308, the feature file is transferred to the interface system at the health care facility.

In the example case of deep learning in computer vision, a feature can be an individual characteristic of what is being observed. In the past, features would be engineered to extract specific things that the researcher thought were relevant to the problem, like a specific colour or a shape. The features can be represented by numbers. However, using deep learning, feature engineering might no longer necessary. The practice of moving away from feature engineering might remove the researcher's bias. For example, making a feature to focus on a specific colour because the researcher thought that it helped detect plants. Instead, the focus can be on engineering architectures, the layers in a neural network, the connections that they form. Initially, the platform 100 is not told what to focus on, so it will learn from scratch whatever is better. If it is the colour green, it will focus on that, or if it is edges, then it will focus on that, and so on.

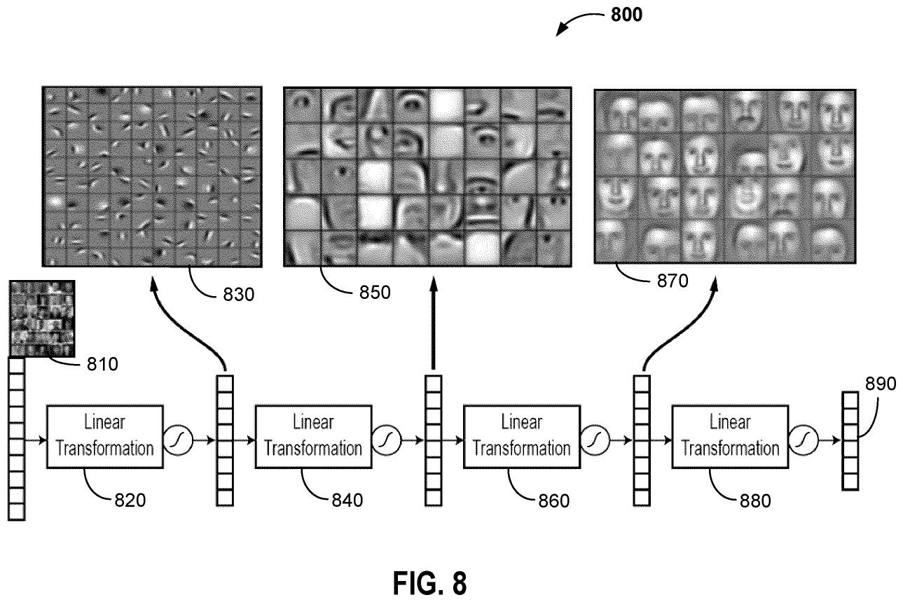

FIG. 8 illustrates an example process 800 of learning features. A neural network can detect where a face is in an image 810. The neural network can include a number of linear transformation sub-processes 820, 840, 860, 880. The detection of a face can be represented by a vector 890 of size 5. For example, the vector can include five elements, each describing, respectively: if a face is presented, height in pixels, width in pixels, centre pixel location on x axis, and centre pixel location on y axis. If there is a face, the value stored at the first position can be 1, if there is no face, the value stored at the first position can be 0, and so on. Each layer in the neural network can be characterised having n filters, with size (h×w). Each of the n filters can go through all the input (e.g., image) applying a linear transformation sub-process 820, 840, 860, 880. The output of all the filters 830, 850, 870 can then be used as the input of the next layer. Once the final layer is reached, the neural network can produce an output. Another function can check if the predicted output matches the actual location of the face, and taking into account the differences, it can go back through all the layers and adjust the linear transformations on the filters. After doing this a number of times, the filters can be specifically adjusted to detect faces. Initially the parameters for the transformations applied by the filters can be set randomly. As the process of training happens the filters learn to detect specific features. An observation of the filters after training can indicate that in the first layer, details or features extracted from an image can be low-level features. For example, the low level features can be edges, contours, little things in multiple orientations shown in example image 830. In middle layers there can be intermediate features, for example in the second layer, intermediate features may look like eyes, eyebrows, nose, mouth, such as those shown in example image 850. In the deeper layers, such as in example image 870, there can be high level features, for example, variations of faces.

The platform 100 can extract different features. For example, there can be high level features from the previous to last layer of the network (e.g., layer 880 before the output). These features can be represented as different numbers. In practice, when visualising features they might not look as neat as in the example picture. The input data can be an image, and instead of obtaining the final result from the last layer, the output of the filters from the previous to last layer can be used as features.

The high level features that the neural network learned can be relevant in the case of head detection.

In the platform 100, the feature vector is then integrated with another network (model 120 architecture), which receives the vector as its input. Then it continues to go through the layers of the architecture and in the last layer it produces an output. This output is a vector that can contain the following: a vector of confidence scores 0-100 (how sure the algorithm is that the detection is a head); a vector of bounding boxes: 2 coordinates, the bottom left and top right of the head in the image. Each vector can have size 40, which means the platform can be able to detect 40 heads at a time (which is likely more than needed for the OR setting). The platform 100 can save the bounding box coordinates for all heads that have a confidence score of a threshold value (e.g. 0.6). The locations of the bounding boxes can be saved to a compressed file. This file is used by the blurring tool 128. It might not be integrated with the video stream at this point.

Once the blurring tool 128 begins generating the blurred video data, it will take as input each frame of the video, load the locations of the bounding boxes, where the heads were detected, for the current frame, and blur the pixels inside the box. This is an example that only the heads in the video are blurred.

At 310, the object detection unit 124 and the blurring tool 128 implement head detection and blurring on a per frame basis. At 312, the platform 100 generates the de-identified file stream. The de-identified file stream includes the blurred video data to blur the images of the faces that were detected in the video data. At 314, the file stream (de-identified) is transferred to the interface system at the health care facility. At 316, file transmission (the file stream, feature file) can be implemented using enterprise secure socket file transfer to a data centre and/or platform 100.

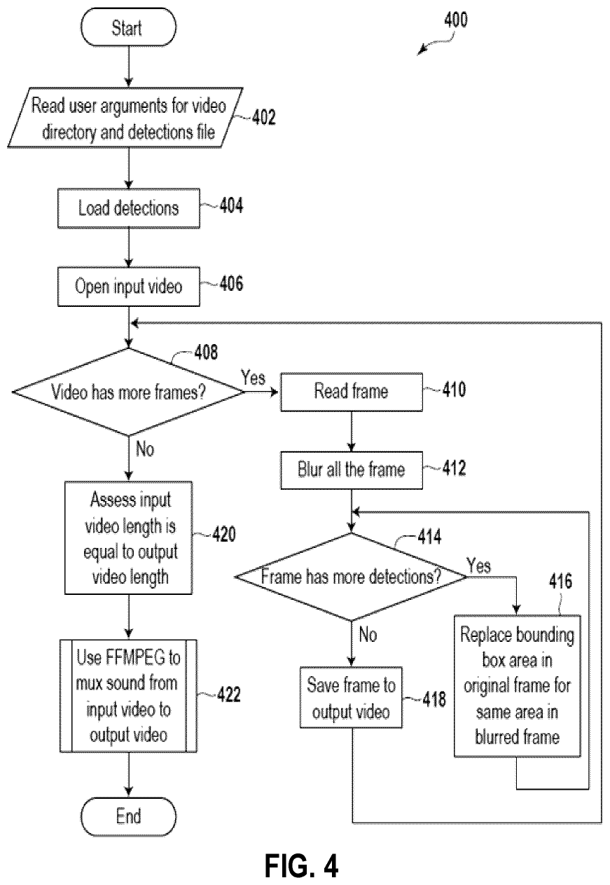

FIG. 4 illustrates a workflow diagram of a process 400 for head blurring in video data according to some embodiments.

At 402, the blurring tool 128 processes the user inputs for the video directory and the location of the detections file. At 404, the detection file is opened, and the frame detection class list is loaded. At 406, the input video is opened. An empty output video is created using the same parameters as the input video. At 408, a loop checks if there are more frames in the video. If there are, at 410, the blurring tool 128 can load the next frame in the video. At 412, the blurring tool 128 can create a blurred copy of this frame. At 414, an inner loop can traverse through each of the detections for the particular frame, and, at 416, the blurring tool 128 can replace the detected head area in the original frame, for the corresponding area in the blurred frame. Once all the detections have been blurred, at 418, the new frame can be saved to the output video. The outer loop, at 408, can check again if there is another frame in the video, until all the frames have been opened. Once all the video has been blurred, at 420, the length of the output video is compared to the length of the input video to make sure that no content was skipped. At 422, a subprocess calls FFMPEG to multiplex the sound from the input video file to the output video file, thus obtaining a blurred video with the soundtrack.

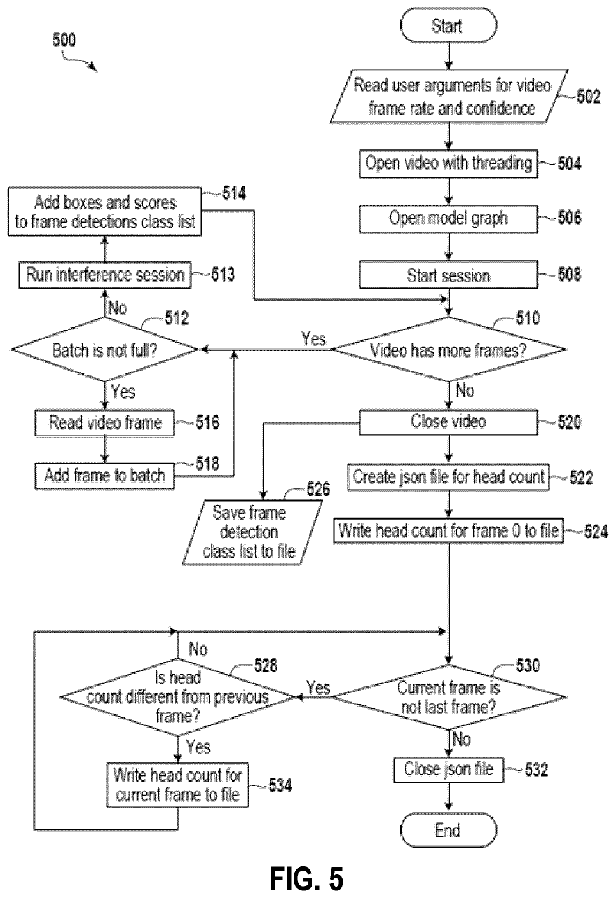

FIG. 5 illustrates a workflow diagram of a process 500 for head detection in video data according to some embodiments.

To start the head detection, at 502, the object detection unit 124 receives as input the directory of the video file, the frame rate to use for the detection, and the threshold confidence rate for the bounding box detections. At 504, the object detection unit 124 can open the video using threading, queueing a number (e.g. 128 as an example) of frames from the video. At 506, the object detection unit 124 loads the graph corresponding to the model that will do the head detection. The model graph can correspond to a frozen version of the model 120. The graph can contain all the linear transformation values for each layer of the model 120. The graph can be frozen because the values in the model 120 will not change. It can work like a black box as it can receive the image as input, apply all the linear transformations for each layer of the model 120, and output the score vector and the bounding box vector. The graph is generated after the model 120 is trained. This is because only with training can there be an adjustment to the linear transformations to perform better for the task, in this case, head detection. Once they have been tweaked, then they can be saved. Mathematically, in every layer there can be many transformations of the form y=wx+b, for example. By training, the platform 100 is adjusting the w's and the b's. By saving it to a graph, the platform 100 can make it easier to load the model 120 to memory and use it, instead of writing it up every time.

At 508, the detection session is started. Within the session, at 510, a while loop can check if there are more frames in the video. If there are more frames, at 512, a number (e.g. 29) of consecutive frames can be stacked together into a batch. At 516, the object detection unit 124 reads the video frame and, at 518, adds the frame to the batch. When the batch is full of frames, at 513, an inference will run on the whole batch of frames. The result from inference will be detection boxes and scores. At 514, the detection boxes and scores can be included into the Frame Detection class list. The loop (510) is repeated until there are no more frames left. Feature extraction is part of the generation of bounding boxes. The platform 100 can have an image of the OR that it feeds into the model 120. The model 120 can be made up of two networks in some embodiments. The first network can extract features, so it can obtain a large representation of the image. The second network can use this large representation to localize where the heads are in the image by generating regions of interest and giving them a score. A number of regions (e.g. 40) with the highest scores can be served as output, giving their coordinates in the image and the confidence score of it being a head. On overview can be: image→[(feature extractor)→(detector)]→(scores)(boxes).

Once all the frames have passed through inference, at 520, the video is closed. At 526, the frame detection class list is saved to a file. At 522, a data file is created that can contain the changes in the head count over the whole video. At 524, the head count for the first frame is added to the file. A loop, at 530, processes each frame until the last frame. At 528, the head count unit 126 can compare the previous frame's head count with the current frame's head count. If the head count has changed, at 534, the new head count and the time corresponding to the frame can be added to the file. Once all the frames are processed, at 532, the file is closed.

Referring back to FIG. 1, the platform 100 includes different models 120 and data sets 122. The models 120 can be modified using different data sets 122, variables, hyperparameters, and learning rates. For example, a model 120 can trained for detection of an example dataset 122 which included 90 classes. For head detection purposes, the platform 100 focuses on one class, head. A First Stage Max proposals can be reduced so that the detections file would be lighter. The max detections and max total detections can reduced to improve the overall speed of the model. The following provides example model variables:
Variable: Original, Modified
Number of Classes: 90, 1
Max total detections: 100, 40
Max detections per class: 100, 40
First Stage Max Proposals: 300, 60.

The model 120 can use different hyperparameters, such as the learning rate, for example. The original learning rate schedule can be: Step 0: 0.0003; Step 900000: 0.00003; Step 1200000: 0.000003. The modified learning rate schedule can be: Step 0: 0.0003; Step 40000: 0.00003; Step 70000: 0.000003.

An example justification for the different learning rates can take into account that the model 120 can be running for 200000 iterations, and that there is only one class being learned in this example data set 122, so convergence can occur faster. Through observation of the precision and recall curves during training, a first plateau was observed around the 35000 to 38000 iterations, which is why the learning rate can be reduced at the 40000 step. Afterwards, a second plateau was observed around step 67000, which is why the second change in the learning rate was made at step 70000. The pre-established learning rates displayed adequate tuning for the purposes of learning a new class. The pre-established learning rates can be maintained.

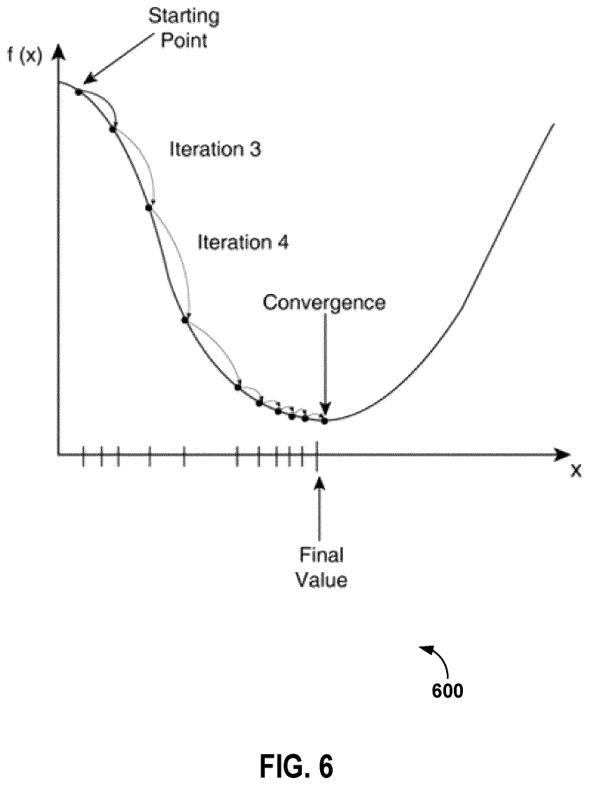

FIG. 6 illustrates an example graph 600 relating to local extrema.

The learning rate describes the size of the step towards the goal. The objective of the algorithm is to minimize the loss, which is why convergence at the lowest point of the curve is desired. The step size can be a variable. A large step size might achieve the goal in less time, but because it is so big, it might not be able to reach the exact minimal value. For example, in training, it might seem like the loss is reducing, and all of a sudden it starts increasing and reducing randomly. This might mean that it is time to reduce the learning rate. This is why the learning rate is reduced after some iterations. This allows for the algorithm to converge, and continue minimizing the loss. There are other ways to change the loss, such as an optimizer that will automatically change the learning rate and the momentum as training is happening (without manual changes).

The platform 100 can train different models 120. In some embodiments, six models 120, varying from the meta-architecture to the feature extractor, can be trained. The model with the best speed/accuracy trade-off can be selected. The frame-rate of the incoming videos can be reduced to 5 fps in order to achieve semi real-time detection. During experiments, the model is able to run on 14 fps, while the cameras capture OR activity at 30 fps. R-FCN model can deliver high accuracy. By reducing the frame rate, speed can be achieved. After the detections are obtained, a script is scheduled to run and process the video data to blur the bounding boxes where the score was higher than 60%. Blurring can be done using different shapes. Some shapes can require more computing power. Blurring can be done using rectangles. Blurring can be done using ellipses. These are examples.

The platform 100 can use models 120 with different architectures.

An example model 120 architecture is faster region-based convolutional neural network (R-CNN). A convolutional neural network (CNN) can be used for image classification. While an R-CNN can be used for object detection (which can include the location of the objects). The R-CNN model 120 is made up by two modules. The first module, called Region Proposal Network (RPN), is a fully convolutional network (FCN) that proposes regions. The second module is made up by the detector (which can be integrated with object detection unit 124). In this model 120, and image will initially go through a feature extraction network, VGG-16, which outputs features that serve as the input to the RPN module. In the RPN, region proposals are generated by sliding a small network over a (n×n) window of the feature map, producing a lower dimensional feature, a maximum of k proposals are generated. Each of the proposals correspond to a reference box or anchor. In some embodiments, the R-CNN model 120 may be a mask R-CNN model, which may use one or more anchor boxes (a set of predefined bounding boxes of a certain height and width) to detect multiple objects, objects of different scales, and overlapping objects in an image. A mask R-CNN can have three types of outputs: a class label and a bounding-box offset for each object, and an object mask. This improves the speed and efficiency for object detection.

Another example model 120 architecture is region-based fully convolutional network (R-FCN). This model 120 is a variation of Faster R-CNN that is fully convolutional and requires lower computation per region proposal. It adopts the two-stage object detection strategy made up of a region proposal and a region classification module. R-FCN extracts features using ResNet-101. Candidate regions of interest (Rols) can extracted by the RPN, while the R-FCN classifies the Rols into object categories, (C+1), and background. The last convolutional layer outputs k2 position-sensitive score maps per category. Finally, R-FCN has a position-sensitive Rol pooling layer which generates scores for each Rol.

Another example model 120 architecture is a single shot multibox detector (SSD). Aiming to make faster detections, SSD uses a single network to predict classes and bounding boxes. It is based on a feed-forward convolutional network that outputs a fixed number of bounding boxes and scores for the presence of a class. Convolutional feature layers in the model enable detections at multiple scales, and produces detection predictions. A non-maximum suppression is applied to produce the final detections. The objective is derived from the detector's objective, while expanded to multiple categories.

Another example model 120 architecture is You Only Look Once (YOLO). This CNN has 24 convolutional layers followed by 2 fully connected layers that predict the output probabilities and coordinates.

As noted, in some embodiments, the head detector process involves feature extraction and generation of a feature vector for each frame. The following neural networks can be used to generate a feature vector from each frame. They will receive the frame, transform it into a vector which will go through the network, coming out as a large feature vector. This feature vector serves as input for the architecture of the model 122.

An example feature extractor is ResNet-101. ResNet reformulates the layers in its network as learning residual functions with reference to the inputs.

Another example feature extractor is Inception v2. This extractor can implement inception units which allow for the increase in the depth and width of a network, while maintaining the computational cost. This can also use batch normalization, which made training faster and regularizes the model, reducing the need for dropout.

Another example feature extractor is Inception-ResNet. This feature extractor is a combination of the inception network with the residual network. This hybrid is achieved by adding a connection to each inception unit. The units can provide a higher computational budget, while the residual connections improve training.

Another example feature extractor is MobileNets. This network was designed for mobile vision applications. The model is built on a factorization of 33 convolutions followed by point-wise convolutions.

An example summary of the models 120 (architecture and feature extractor) is as follows: (Mobilenets; SSD); (Inception v2; SSD); (Resnet101; R-FCN); (Resnet101; Faster R-CNN); (Inception-Resnet; Faster R-CNN).

The feature vector for each frame can be used to increase the number of elements visible to the neural network. An example can use ResNet101. It takes in a small image as input (e.g. 224×224 pixels in size). This image has three channels, R,G,B. so its size is actually 224×224×3. Oversimplifying the image, we have 150528 pixel values. These describe colour only. This is ResNets input, after the first block of convolution transformations we will have a 64×64×256 volume, so that is 1048576 different values. And these values not only describe colours, but also edges, contours, and other low level features. After the second convolutional block, an output volume of 128×128×512 is obtained, corresponding to 8388608 values, of a little higher level features than the last block. In the next block a 256×256×1024 (67108864) is obtained, higher level features than before. And in the next layer we obtain a 512×512×2048 (536870912) volume is obtained, with higher level features.

In summary, the objective is to increase the description of the input to the network (model 120), so instead of having just a 224×224×3 image, with numerical description of only colours, we now have a 512×512×2048 volume, that means we have increased the number of values (features) we input to the network by 3566 times. The features that describe the input are not only colours but, anything the network learned to detect that is useful when describing images with heads. This might be caps, eyes, masks, facial hair, ears. The feature vector is big compared to the initial image, so it can be referred to as a large feature vector.

FIG. 7 illustrates a schematic of an architectural platform 10 for data collection in a live OR setting or patient intervention area according to some embodiments. Further details regarding data collection and analysis are provided in International (PCT) Patent Application No. PCT/CA2016/000081 entitled “OPERATING ROOM BLACK-BOX DEVICE, SYSTEM, METHOD AND COMPUTER READABLE MEDIUM FOR EVENT AND ERROR PREDICTION” and filed Mar. 26, 2016 and International (PCT) Patent Application No. PCT/CA2015/000504, entitled “OPERATING ROOM BLACK-BOX DEVICE, SYSTEM, METHOD AND COMPUTER READABLE MEDIUM” and filed Sep. 23, 2015, the entire contents of each of which is hereby incorporated by reference.

The data collected relating to the OR activity and be correlated and/or synchronized with other data collected from the live OR setting by the platform 10. For example, a number of individuals participating in a surgery can be linked and/or synchronized with other data collected from the live OR setting for the surgery. This can also include data post-surgery, such as data related to the outcome of the surgery.

The platform 10 can collect raw video data for processing in order to detect heads as described herein. The output data (head detection and count estimates) can be aggregated with other data collected from the live OR setting for the surgery or otherwise generated by platform 10 for analytics.

The platform 10 includes various hardware components such as a network communication server 12 (also “network server”) and a network control interface 14 (including monitor, keyboard, touch interface, tablet, processor and storage device, web browser) for on-site private network administration.

Multiple processors may be configured with operating system and client software (e.g., Linux, Unix, Windows Server, or equivalent), scheduling software, backup software. Data storage devices may be connected on a storage area network.

The platform 10 can include a surgical or medical data encoder 22. The encoder may be referred to herein as a data recorder, a “black-box” recorder, a “black-box” encoder, and so on. Further details will be described herein. The platform 10 may also have physical and logical security to prevent unintended or unapproved access. A network and signal router 16 connects components.

The platform 10 includes hardware units 20 that include a collection or group of data capture devices for capturing and generating medical or surgical data feeds for provision to encoder 22. The hardware units 20 may include cameras 30 (e.g. including cameras for capturing video of OR activity) internal to patient to capture video data for provision to encoder 22. The encoder 22 can implement the head detection and count estimation described herein in some embodiments. The video feed may be referred to as medical or surgical data. An example camera 30 is a laparoscopic or procedural view camera resident in the surgical unit, ICU, emergency unit or clinical intervention units. Example video hardware includes a distribution amplifier for signal splitting of Laparoscopic cameras. The hardware units 20 can have audio devices 32 mounted within the surgical unit, ICU, emergency unit or clinical intervention units to provide audio feeds as another example of medical or surgical data. Example sensors 34 installed or utilized in a surgical unit, ICU, emergency unit or clinical intervention units include but not limited to: environmental sensors (e.g., temperature, moisture, humidity, etc., acoustic sensors (e.g., ambient noise, decibel), electrical sensors (e.g., hall, magnetic, current, mems, capacitive, resistance), flow sensors (e.g., air, fluid, gas) angle/positional/displacement sensors (e.g., gyroscopes, altitude indicator, piezoelectric, photoelectric), and other sensor types (e.g., strain, level sensors, load cells, motion, pressure). The sensors 34 provide sensor data as another example of medical or surgical data. The hardware units 20 also include patient monitoring devices 36 and an instrument lot 18.

The customizable control interface 14 and GUI (may include tablet devices, PDA's, hybrid devices, convertibles, etc.) may be used to control configuration for hardware components of unit 20. The platform 10 has middleware and hardware for device-to-device translation and connection and synchronization on a private VLAN or other network. The computing device may be configured with anonymization software, data encryption software, lossless video and data compression software, voice distortion software, transcription software. The network hardware may include cables such as Ethernet, RJ45, optical fiber, SDI, HDMI, coaxial, DVI, component audio, component video, and so on to support wired connectivity between components. The network hardware may also have wireless base stations to support wireless connectivity between components.

The platform 10 can include anonymization software for anonymizing and protecting the identity of all medical professionals, patients, distinguishing objects or features in a medical, clinical or emergency unit. This software implements methods and techniques to detect facial, distinguishing objects, or features in a medical, clinical or emergency unit and distort/blur the image of the distinguishing element. The extent of the distortion/blur is limited to a localized area, frame by frame, to the point where identity is protected without limiting the quality of the analytics. The software can be used for anonymizing the video data as well.

Data encryption software may execute to encrypt computer data in such a way that it cannot be recovered without access to the key. The content may be encrypted at source as individual streams of data or encrypted as a comprehensive container file for purposes of storage on an electronic medium (i.e. computer, storage system, electronic device) and/or transmission over internet 26. Encrypt/decrypt keys may either be embedded in the container file and accessible through a master key, or transmitted separately.

Lossless video and data compression software executes with a class of data compression techniques that allows the original data to be perfectly or near perfectly reconstructed from the compressed data.

Device middleware and hardware may be provided for translating, connecting, formatting and synchronizing of independent digital data streams from source devices. The platform 10 may include hardware, software, algorithms and methods for the purpose of establishing a secure and reliable connection and communication directly, or indirectly (via router, wireless base station), with the OR encoder 22, and third-party devices (open or proprietary) used in a surgical unit, ICU, emergency or other clinical intervention unit.

The hardware and middleware may assure data conformity, formatting and accurate synchronization. Synchronization may be attained by utilizing networking protocols for clock synchronization between computer systems and electronics devices over packet-switched networks like NTP, etc.

The encoder 22 can implement the head detection and count estimation described herein in some embodiments. The encoder 22 can provide video data and other data to another server for head detection and count estimation described herein in some embodiments. The OR or Surgical encoder (e.g., encoder 22) may be a multi-channel encoding device that records, integrates, ingests and/or synchronizes independent streams of audio, video, and digital data (quantitative, semi-quantitative, and qualitative data feeds) into a single digital container. The digital data may be ingested into the encoder as streams of metadata and is sourced from an array of potential sensor types and third-party devices (open or proprietary) that are used in surgical, ICU, emergency or other clinical intervention units. These sensors and devices may be connected through middleware and/or hardware devices which may act to translate, format and/or synchronize live streams of data from respected sources.

The Control Interface (e.g., 14) may include a Central control station (non-limiting examples being one or more computers, tablets, PDA's, hybrids, and/or convertibles, etc.) which may be located in the clinical unit or another customer designated location. The Customizable Control Interface and GUI may contain a customizable graphical user interface (GUI) that provides a simple, user friendly and functional control of the system.

The encoder 22 may be responsible for synchronizing all feeds, encoding them into a signal transport file using lossless audio/video/data compression software. Upon completion of the recording, the container file will be securely encrypted. Encrypt/decrypt keys may either be embedded in the container file and accessible through a master key, or transmitted separately. The encrypted file may either be stored on the encoder 22 or stored on a Storage area network until scheduled transmission.

According to some embodiments, this information then may be synchronized (e.g., by the encoder 22) and/or used to evaluate: technical performance of the healthcare providers; non-technical performance of the clinical team members; patient safety (through number of registered errors and/or adverse events); occupational safety; workflow; visual and/or noise distractions; and/or interaction between medical/surgical devices and/or healthcare professionals, etc. According to some embodiments, this may be achieved by using objective structured assessment tools and questionnaires and/or by retrieving one or more continuous data streams from sensors 34, audio devices 32, an anesthesia device, medical/surgical devices, implants, hospital patient administrative systems (electronic patient records), or other data capture devices of hardware unit 20. According to some embodiments, significant “events” may be detected, tagged, time-stamped and/or recorded as a time-point on a timeline that represents the entire duration of the procedure and/or clinical encounter. The timeline may overlay captured and processed data to tag the data with the time-points. In some embodiments, the events may be head detection events or count events that exceed a threshold number of people in the OR.

Upon completion of data processing and analysis, one or more such events (and potentially all events) may be viewed on a single timeline represented in a GUI, for example, to allow an assessor to: (i) identify event clusters; (ii) analyze correlations between two or more registered parameters (and potentially between all of the registered parameters); (iii) identify underlying factors and/or patterns of events that lead up to adverse outcome; (iv) develop predictive models for one or more key steps of an intervention (which may be referred to herein as “hazard zones”) that may be statistically correlated to error/adverse event/adverse outcomes, v) identify a relationship between performance outcomes and clinical costs. These are non-limiting examples of uses an assessor may make of a timeline presented by the GUI representing recorded events.

Analyzing these underlying factors according to some embodiments may allow one or more of: (i) proactive monitoring of clinical performance; and/or (ii) monitoring of performance of healthcare technology/devices (iii) creation of educational interventions—e.g., individualized structured feedback (or coaching), simulation-based crisis scenarios, virtual-reality training programs, curricula for certification/re-certification of healthcare practitioners and institutions; and/or identify safety/performance deficiencies of medical/surgical devices and develop recommendations for improvement and/or design of “intelligent” devices and implants—to curb the rate of risk factors in future procedures and/or ultimately to improve patient safety outcomes and clinical costs.

The device, system, method and computer readable medium according to some embodiments, may combine capture and synchronization, and secure transport of video/audio/metadata with rigorous data analysis to achieve/demonstrate certain values. The device, system, method and computer readable medium according to some embodiments may combine multiple inputs, enabling recreation of a full picture of what takes place in a clinical area, in a synchronized manner, enabling analysis and/or correlation of these factors (between factors and with external outcome parameters (clinical and economical). The system may bring together analysis tools and/or processes and using this approach for one or more purposes, examples of which are provided herein.

Beyond development of a data platform 10, some embodiments may also include comprehensive data collection and/or analysis techniques that evaluate multiple aspects of any procedure including video data of OR procedures and participants. One or more aspects of embodiments may include recording and analysis of video, audio and metadata feeds in a synchronized fashion. The data platform 10 may be a modular system and not limited in terms of data feeds—any measurable parameter in the OR/patient intervention areas (e.g., data captured by various environmental acoustic, electrical, flow, angle/positional/displacement and other sensors, wearable technology video/data stream, etc.) may be added to the data platform 10. One or more aspects of embodiments may include analyzing data using validated rating tools which may look at different aspects of a clinical intervention.

According to some embodiments, all video feeds and audio feeds may be recorded and synchronized for an entire medical procedure. Without video, audio and data feeds being synchronized, rating tools designed to measure the technical skill and/or non-technical skill during the medical procedure may not be able to gather useful data on the mechanisms leading to adverse events/outcomes and establish correlation between performance and clinical outcomes.

According to some embodiments, measurements taken (e.g., error rates, number of adverse events, individual/team/technology performance parameters) may be collected in a cohesive manner. According to some embodiments, data analysis may establish correlations between all registered parameters as appropriate. With these correlations, hazard zones may be pinpointed, high-stakes assessment programs may be developed and/or educational interventions may be designed.

Experimental results are presented below by de-identifying facial features on a human head using a system described herein, for example, using platform 100. To perform the evaluation, a five-second video clip is chosen from three different hospital sites: Hospital Site 1, Hospital Site 2, and Hospital Site 3. All of the video clips are from surgeries performed in real life with visible patients and surgical team members. Each video clip has been processed by the system using one two detection types: “head” and “patient”. Example system has been trained on videos from Hospital Site 2 and Hospital Site 3, which provide the test data set. Hospital Site 1 is considered a source of transfer learning test data set.

| site | #camera | #case | #clip |
|---|---|---|---|
| Hospital Site 1 | 2 | 9 | 18 |
| Hospital Site 2 | 3 | 9 | 27 |
| Hospital Site 3 | 2 | 9 | 18 |

In order to de-identify facial features of people in the operating room, the system may implement a blurring tool 128 with a R-FCN model to blur the facial features or head of the people in the operating room. The R-FCN model may be: 1) pre-trained on MS COCO (Microsoft Common Objects in Context) dataset, which is a large-scale object detection, segmentation, key-point detection, and captioning dataset; 2) finetuned on a proprietary Head Dataset (cases from various hospitals); and 3) finetuned on a proprietary Head+Patient Dataset (cases from various hospitals such as Hospital Site 2 and Hospital Site 3).

Evaluation on head blurring uses metric of combined manner of detection and mask. For one frame, how much percentage of a region of interest (ROI) is covered by true positive indications is evaluated.

The following metrics are observed throughout the experiments:
Recall: indicates how many ground truth are detected. For one frame, if the pixel percentage of (true positive/ground truth) is over the threshold, recall on this frame is set to the percentage; otherwise, it is set to 0;
Precision: indicates how many detection are real heads. For one frame, if the pixel percentage of (true positive/positive) is over the threshold, precision on this frame is set to the percentage; otherwise, it is set to 0;
Lower thresholds can have higher recall/precision;
Overall recall/precision is averaged among all frames; and
Multi-class is evaluated separately.

Considering the problem definition and method of detection, some changes to the way of calculating of recall and precision as the meaning of coverage:
recall: true positive coverage rate: pixel percentage for each frame is calculated as 0.8 (while traditional recall determines the value as 1 if over threshold), and then average on frames; and
precision: positive predictive coverage rate: pixel percentage for each frame is calculated as 0.8 (while traditional recall determines the value as 1 if over threshold), and then average frames.

That is, true percentage on each frame, lower than commonly-used 1 is used. Specifically, note that recall=0.8 does not mean 20% of objects are missed.

Intersection threshold shows how strictly when comparing prediction and ground truth. The values vary from 0 to 1.

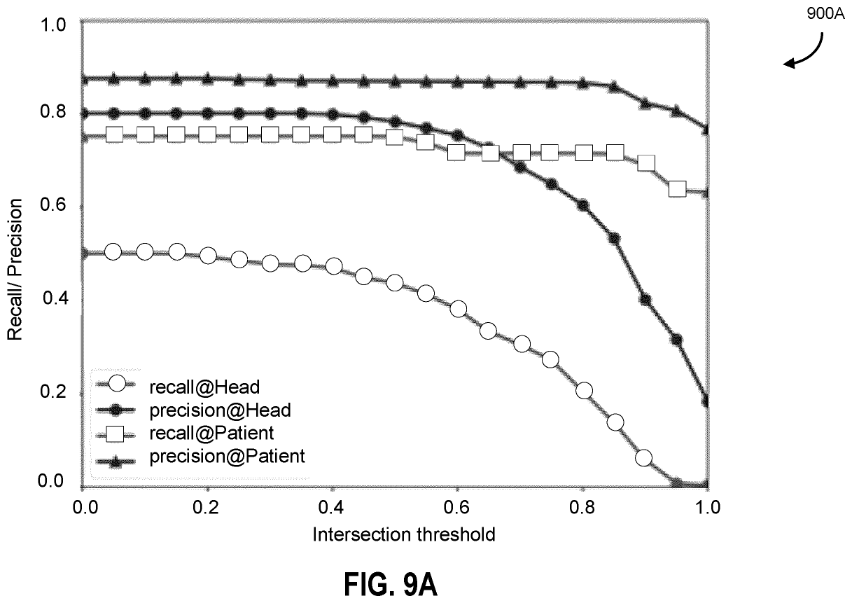

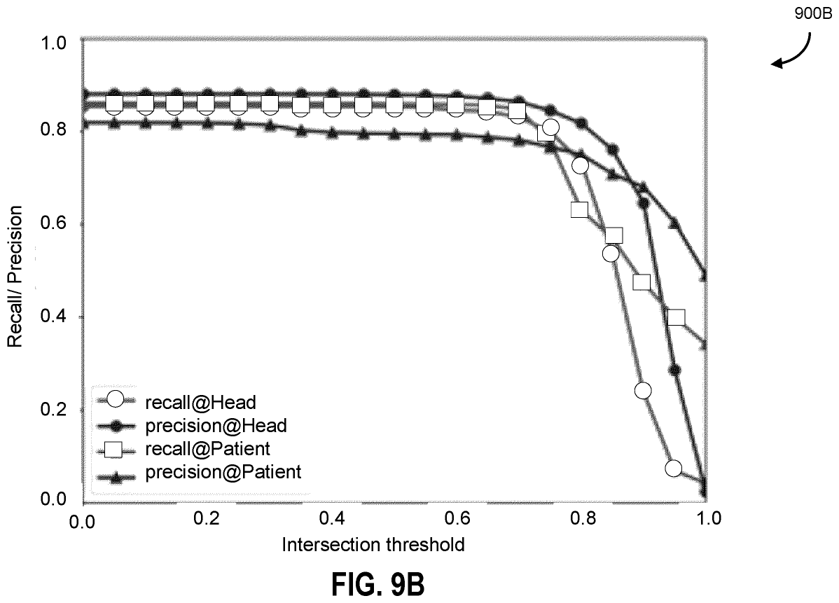

FIG. 9A is a graph 900A that illustrates experimental results of an example system used to de-identify features from a video obtained at a first hospital site (Hospital Site 1). FIG. 9B is a graph 900B illustrates experimental results of an example system used to de-identify features in from a video obtained at a second hospital site Hospital Site 2. These experimental results are obtained on setting of sampling_rate=5, batch_size=24.

As can be seen from FIGS. 9A and 9B, for commonly used thresholds 0.5 and 0.75, and the disclosed model behaves basically similar on these for Hospital Site 2 and Hospital Site 3, meaning predicted bounding boxes are precise.

When threshold goes the 1.0, some of recalls/precisions are not approaching 0. This is because for frames having no head or patient, the frames may be marked as 1 for both recall and precision (meaning this frame has been de-identified properly). Results from Hospital Site 1 are worse than Hospital Site 2/Hospital Site 3, meaning site-specific fine-tuning may be necessary.

In some experiments, only K key frames are sampled and detected on the input videos, with some frames skipped between the key frames. Using a sampling approach may not affect results when people stay still (most cases) but the result may be inferior when people move fast in the operating room in a given time frame.

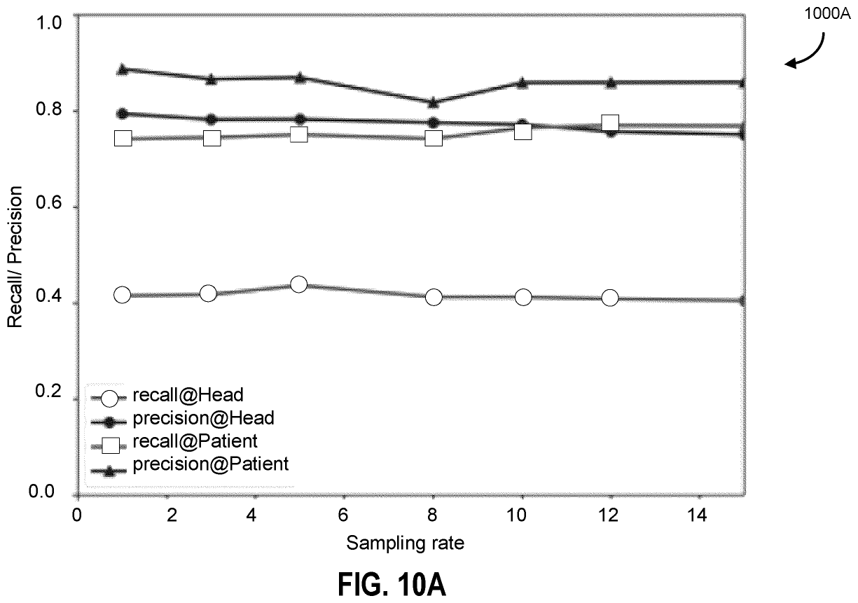

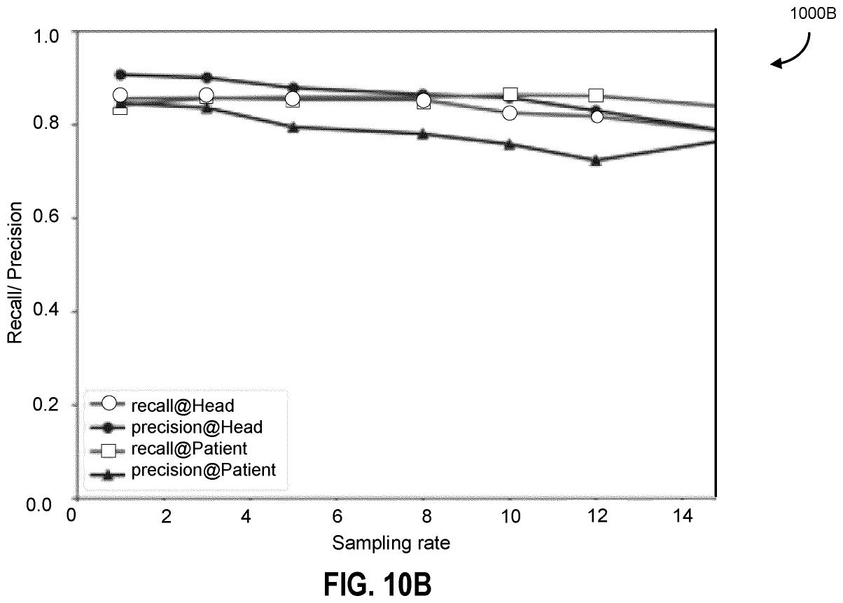

FIG. 10A is a graph 1000A that illustrates experimental results of an example system used to de-identify features from the video obtained at a first hospital site (Hospital Site 1) using different sampling rates. FIG. 10B is a graph 1000B that illustrates experimental results of an example system used to de-identify features in from a video obtained at a second hospital site (Hospital Site 2) using different sampling rates. The value for K may vary from 1 to 15. This is evaluated on setting of P/R threshold=0.5, batch_size=24.

As can be seen from FIGS. 10A and 10B, missed detection is fixed by adding smoothing at beginning/end of each trajectory. Sampling less and missing a few will not impact the performance significantly, and can be fixed by momentum-based smoothing. Smoothing is added at beginning/end with K key frames. When the sampling rate is larger, the number of smoothed frames is greater, fixing is better, recall is higher, and precision is lower. Momentum is calculated by average speed at beginning/end. When the sampling rate is larger, the momentum is more stable, and the trajectory is more smooth. In addition, fixing can fail when momentum cannot represent the missing movement, especially when acceleration is higher, such as when a direction of one or more objects is changed quickly, or when one or more objects are moving or accelerating at a rate that is above a threshold.

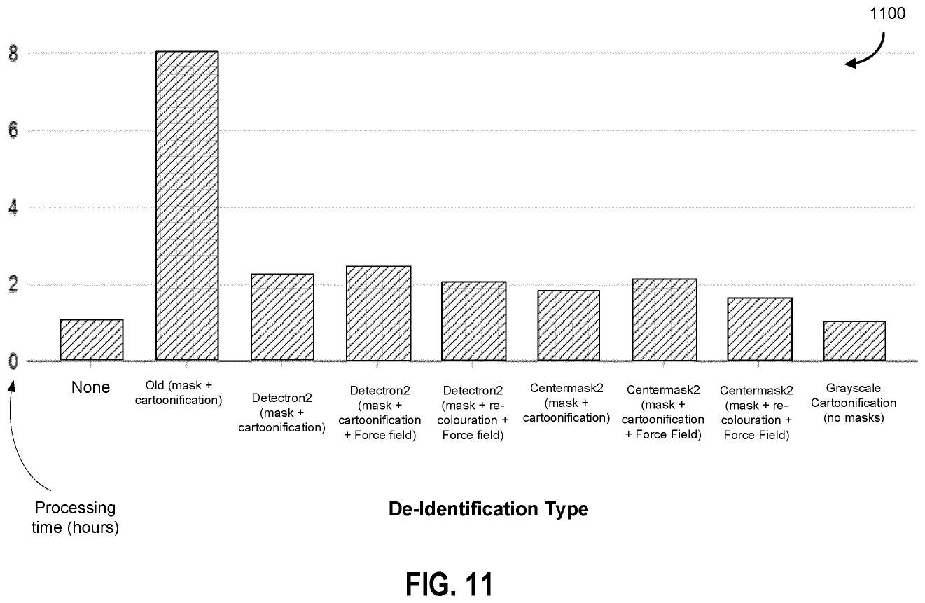

FIG. 11 is a graph 1100 that illustrates example processing time of various de-identification types in hours in a first chart, for both head and body. As can be seen, prior methods with mask and cartoon-ification takes significantly more time than Detectron2 and Centermask2 methods.

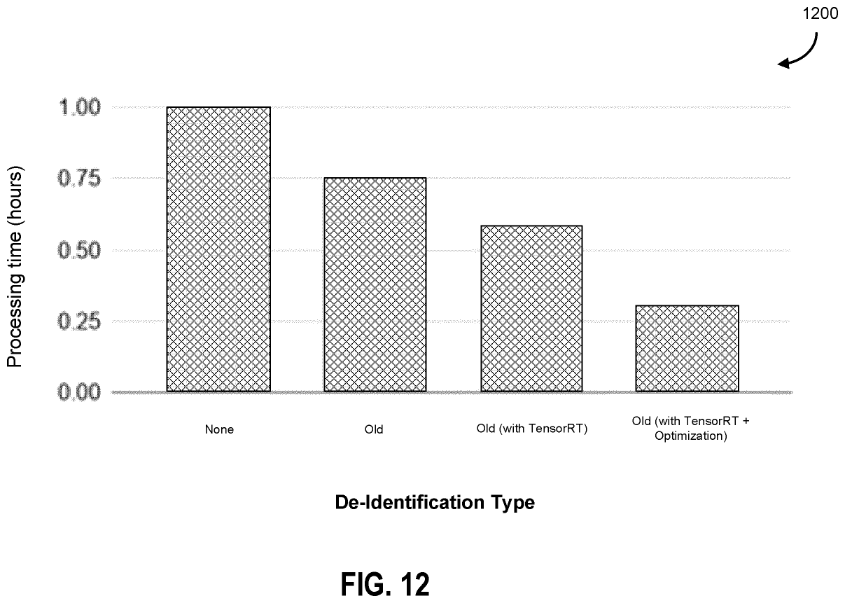

FIG. 12 is a graph 1200 that illustrates example processing time of various de-identification types in hours in a second chart. As can be seen, tensorRT performs better (less time) with optimisation than without optimisation.

For false negative rate (FNR), the experimental results obtained from Hospital Site 1 video has a FNR of 0.24889712, the experimental results obtained from Hospital Site 2 video has a FNR of 0.14089696, and the experimental results obtained from Hospital Site 3 video has a FNR of 0.21719834.

For false discovery rate (FDR), the experimental results obtained from Hospital Site 1 video has a FDR of 0.130006, the experimental results obtained from Hospital Site 2 video has a FDR of 0.20503037, and the experimental results obtained from Hospital Site 3 video has a FDR of 0.09361056.

The discussion herein provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the discussion herein, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein. Moreover, the scope of the present application is not intended to be limited to the particular embodiments of the process, machine, manufacture, composition of matter, means, methods and steps described in the specification. As can be understood, the examples described above and illustrated are intended to be exemplary only.

1. A computer-implemented method for traffic monitoring in an operating room, the method comprising:
receiving video data of an operating room, the video data captured by a camera having a field of view for viewing movement of a plurality of individuals in the operating room during a medical procedure;
storing an event data model including data defining a plurality of possible events within the operating room, the event data model trained to utilize a total number of people in the operating room as a conditioning variable, and trained using a data set of training frames from training procedures used to update a set of weight parameters representing a trained event data model;
processing the video data using one or more trained object-tracking models configured to track movement of objects within the operating room, the objects including at least one body part, and the processing using at least one detector trained to detect a given type of the objects, the movement of the objects processed to estimate changes to a total number of people in the operating room based on the at least one body part in the video data at a particular time and to record a timestamp when the estimated change occurs; and
at a particular time or duration of time, determining a likelihood of occurrence of one of the possible events based on the tracked movement and by processing the estimated total number of people in the operating room as the conditioning variable against the trained event data model; and
generating a data output representative of the likelihood of occurrence of one of the possible events.

2. The computer-implemented method of claim 1, wherein the at least one body part includes at least one of a limb, a hand, a head, or a torso.

3. The computer-implemented method of claim 1, wherein the plurality of possible events includes adverse events.

4. The computer-implemented method of claim 1, wherein the video data is processed to generate a first output file that records timestamped changes to the estimated total number of people in the operating room, and to generate a second output file containing bounding boxes of each detected object, a confidence score of detection of the detected object, and a frame number upon which the detected object is visible.

5. The computer-implemented method of claim 1, wherein determining the likelihood of occurrence of one of the possible events includes determining that the count of people exceeds a pre-defined threshold.

6. The computer-implemented method of claim 4, wherein a separate object-tracking model of a plurality of object-tracking models is utilized to detect different types of objects in the operating room.

7. The computer-implemented method of claim 4, wherein the count describes a number of individuals in a portion of the operating room, the portion of the operating room defined using a stored floorplan data structure including data and metadata that describes a floorplan or layout of at least a portion of the operating room such that positions of objects are estimated with reference to a three-dimensional coordinate system relative to the operating room, and wherein the determination of the likelihood of occurrence of one of the possible events based on the tracked movement includes providing the estimated positions of objects to the trained event data model.

8. The computer-implemented method of claim 1, further comprising determining a correlation between the likely occurrence of one of the possible events and a distraction.

9. The computer-implemented method of claim 1, wherein the one or more object-tracking models are trained the data set of training frames from training procedures used to update the set of weight parameters representing the trained event data model include variations with at least one of occlusions, different colored caps, and masks, and
wherein the trained model and its weights are exported to a inference graph data object objects include a device within the operating room.

10. The computer-implemented method of claim 9, wherein the object-tracing models are trained to track a proximity of an object relative to device is a radiation-emitting device.

11-16. (canceled)

17. A computer system for monitoring traffic in an operating room, the system comprising:
at least one processor;
memory in communication with said at least one processor; and
software code stored in said memory, which when executed at the at least one processor causes said system to:
receive video data of an operating room, the video data captured by a camera having a field of view for viewing movement of a plurality of individuals in the operating room during a medical procedure;
store an event data model including data defining a plurality of possible events within the operating room;
process the video data to track movement of objects within the operating room, the objects including at least one body part, and the processing using at least one detector trained to detect a given type of the objects; and
determine a likely occurrence of one of the possible events based on the tracked movement.

18. The system of claim 17, wherein the at least one body part includes at least one of a limb, a hand, a head, or a torso.

19. The system of claim 17, wherein the plurality of possible events includes adverse events.

20. The system of claim 17, wherein the at least one processor causes said system to further determine a count of individuals based on the processing using at least one detector.

21. The system of claim 17, wherein determining a likely occurrence of one of the possible events includes determining that the count of individuals exceeds a pre-defined threshold.

22. The system of claim 20, wherein the count describes a number of individuals in the operating room.

23. The system of claim 20, wherein the count describes a number of individuals in a portion of the operating room.

24. The system of claim 17, wherein the at least one processor causes said system to further determine a correlation between the likely occurrence of one of the possible events and a distraction.

25-32. (canceled)

33. A non-transitory computer-readable storage medium storing machine executable instructions which when executed by a processor, cause the processor to perform a method for traffic monitoring in an operating room, the method comprising:
receiving video data of an operating room, the video data captured by a camera having a field of view for viewing movement of a plurality of individuals in the operating room during a medical procedure;
storing an event data model including data defining a plurality of possible events within the operating room, the event data model trained to utilize a total number of people in the operating room as a conditioning variable, and trained using a data set of training frames from training procedures used to update a set of weight parameters representing a trained event data model;
processing the video data using one or more trained object-tracking models configured to track movement of objects within the operating room, the objects including at least one body part, and the processing using at least one detector trained to detect a given type of the objects, the movement of the objects processed to estimate changes to a total number of people in the operating room based on the at least one body part in the video data at a particular time and to record a timestamp when the estimated change occurs; and
at a particular time or duration of time, determining a likelihood of occurrence of one of the possible events based on the tracked movement and by processing the estimated total number of people in the operating room as the conditioning variable against the trained event data model; and
generating a data output representative of the likelihood of occurrence of one of the possible events.

34. The non-transitory computer-readable storage medium of claim 33, wherein the non-transitory computer-readable storage medium operates on a programmable computer connected to one or more data sources across a network that is coupled to the camera for receiving the video data from the camera, the programmable computer configured to maintain trained model architectures for object detection including at least the one or more trained object-tracking models, the trained model architectures including deep learning models trained using a data set constructed from video feeds including random frames taken from self-recorded procedures in the operating room and bounding-box annotations around heads of people in the operating room, and the head count unit for computing head count data based on the detected heads in the video data.
